(19) United States
(12) Patent Application Publication
(10) Pub. No.: US 2021/0401243 A1
(43) Pub. Date: Dec. 30, 2021

(54) DRYING SYSTEM, INFORMATION PROCESSING APPARATUS, AND NON-TEMPORARY STORAGE MEDIUM

(71) Applicant: TOYOTA JIDOSHA KABUSHIKI KAISHA, Toyota-shi (JP)

(72) Inventors: Yurika TANAKA, Yokosuka-shi (JP); Shuichi SAWADA, Nagoya-shi (JP); Shin SAKURADA, Toyota-shi (JP); Yasuhiro BABA, Kamo-gun (JP); Shintaro MATSUTANI, Kariya-shi (JP); Tomoya MAKINO, Kariya-shi (JP)

(73) Assignee: TOYOTA JIDOSHA KABUSHIKI KAISHA, Toyota-shi (JP)

(21) Appl. No.: 17/354,459

(22) Filed: Jun. 22, 2021

(30) Foreign Application Priority Data
Jun. 29, 2020 (JP) 2020-111468

(51) Int. Cl. A47K 10/48; A47K 7/00

(52) U.S. Cl. CPC A47K 10/48; G06K 9/00375; A47K 7/00

(57) ABSTRACT

According to the present disclosure, a user is effectively notified of whether hand-washing is sufficient or not.

A drying system according to the present disclosure includes a first sensor configured to sense a washed hand of a user and a controller configured to acquire first information on the temperature of the washed hand of the user based on data acquired from the first sensor and determine whether to provide a dry medium for the user based on the first information.

### CROSS REFERENCE TO THE RELATED APPLICATION

This application claims the benefit of Japanese Patent Application No. 2020-111468, filed on Jun. 29, 2020, which is hereby incorporated by reference herein in its entirety.

### BACKGROUND

### Technical Field

The present disclosure relates to a technique for supporting hand-washing.

### Description of the Related Art

Hand-washing is practiced to prevent infectious diseases. Hand-washing in a sufficient time is preferable for removing viruses. For example, Japanese Patent Laid-Open No. 2020-000651 discloses an apparatus for notifying a user of a difference between an intended hand-washing time and an actual hand-washing time as related art.

[Patent document 1] Japanese Patent Laid-Open No. 2020-000651

### SUMMARY

Existing techniques for hand-washing has still been in need of improvements in accurate determination on whether hand-washing is sufficient or not and effective notification on the determination to a user.

One or more aspects of the present disclosure are directed to provide a technique of effectively notifying a user of whether hand-washing is sufficient or not.

A first aspect of the present disclosure may be a drying system including a first sensor configured to sense a washed hand of a user and a controller including at least one processor configured to acquire first information on the temperature of the washed hand of the user based on data acquired from the first sensor and determine whether to provide a dry medium for the user based on the first information.

A second aspect of the present disclosure may be an information processing apparatus including a controller including at least one processor configured to acquire first information on the temperature of the washed hand of a user based on data acquired from a first sensor configured to sense the washed hand of the user, and determine whether to provide a dry medium for the user based on the first information.

A third aspect of the present disclosure may be a non-temporary storage medium for storing a program for causing an information processing apparatus to acquire first information on the temperature of the washed hand of a user based on data acquired from a first sensor configured to sense the washed hand of the user, and determine whether to provide a dry medium for the user based on the first information.

Another aspect may be an information processing method performed by the information processing apparatus.

According to the present disclosure, a user can be effectively notified of whether hand-washing is sufficient.

### BRIEF DESCRIPTION OF THE DRAWINGS

FIG. 1 is an explanatory drawing illustrating the outline of a dryer according to an embodiment;

FIG. 2 is a schematic diagram illustrating the configuration of the dryer according to a first embodiment;

FIG. 3 is an explanatory drawing of a user's hand to be sensed;

FIG. 4A and FIG. 4B are explanatory drawings illustrating the distribution of a calculated surface temperature;

FIG. 5 is a flowchart of processing performed by a controller according to the first embodiment;

FIG. 6 is a schematic diagram illustrating the configuration of a drying system according to a second embodiment;

FIG. 7 is a flowchart of processing performed by a controller according to the second embodiment;

FIG. 8 is an explanatory drawing indicating a change of a temperature distribution before and after hand-washing;

FIG. 9 is a schematic diagram illustrating the configuration of a drying system according to a third embodiment;

FIG. 10 is an explanatory drawing indicating the timing of supply of water and soap;

FIG. 11 is a flowchart of processing performed by a controller according to the third embodiment; and

FIG. 12 is a schematic diagram illustrating the configuration of a dryer according to a fourth embodiment.

### DESCRIPTION OF THE EMBODIMENTS

Apparatuses for assisting hand-washing of users are known. For example, in order to teach exemplary washing time and operations to a user, an apparatus outputs related information. Unfortunately, such an apparatus can output auxiliary information but includes difficulty in determining whether user's hands have been actually cleaned.

A drying system for solving the problem according to the present embodiment may include a first sensor configured to sense a washed hand of a user and a controller configured to acquire first information on the temperatures of the washed hand of the user based on data acquired from the first sensor and determine whether to provide a dry medium for the user based on the first information.

Generally, a finger temperature changes during hand-washing in water depending on a hand-washing time. For example, hand-washing in cold water may cause a finger temperature to fall below a body temperature. Moreover, as the hand-washing time increases, a reduction in temperature becomes larger. Thus, information on the temperature of a washed hand of a user can be acquired to determine whether fingers have been washed in sufficient hand-washing water, and thereby estimate whether hand-washing has been performed for a sufficient time. Furthermore, whether to provide a dry medium is determined based on the determination result, thereby effectively notifying a user of whether hand-washing is sufficient or not.

The first information is typically information on the surface temperature of a hand. The first information may be any information on the temperature of a user's hand. The first sensor is preferably, for example, a sensor capable of measuring temperature in a noncontact manner.

The dry medium is typically warm air. Any medium for removing moisture may be used instead. For example, the dry medium may be cold air, a paper towel, or a cloth towel.

The controller may determine that the dry medium is to be provided when the hand temperature is lower than a predetermined value.

The hand temperature is typically a temperature on the skin surface of a hand. In the case of temperature measurement at multiple points, a determination may be made using values calculated from temperatures at the multiple points.

The first sensor may be a sensor capable of acquiring the temperature distribution of the hand, the first information may be information on the temperature distribution of the hand, and the controller may determine whether to provide the dry medium based on the temperature distribution of the hand.

The controller may determine that the dry medium is to be provided when an evaluation value calculated from the temperature distribution of the hand satisfies a predetermined threshold value.

The acquisition of the temperature distribution of the hand can determine whether the hand has been entirely washed in water.

The drying system may further include a second sensor configured to sense a hand of a user before the hand-washing, wherein the controller may acquire second information on the temperature of the hand of the user before the hand-washing based on data acquired from the second sensor and determine whether to provide the dry medium based on both of the first information and the second information.

The second sensor is a sensor for sensing a hand of a user before hand-washing. The second sensor may be installed at, for example, a washstand. The second sensor may also act as a sensor for an automatic water tap. The use of the second information can determine a temperature change in a user's hand before and after hand-washing. This can reduce the influence of the body temperature of a user and an air temperature.

The first sensor and the second sensor may be sensors capable of acquiring the temperature distribution of the hand, the first information and the second information may be information on the temperature distribution of the hand, and the controller may determine whether to provide the dry medium based on a change of the temperature distribution of the hand.

In this way, a change of the temperature distribution of a user's hand before and after hand-washing is obtained by using the second sensor, enabling a determination with higher accuracy.

The drying system may further include a storage configured to store criteria for determining whether to provide the dry medium.

The storage can store criteria, for example, conditions to be satisfied by the obtained temperature distribution in order to provide the dry medium or a change to be made by the temperature distribution in order to provide the dry medium.

The drying system may further include a third sensor configured to obtain a water temperature of water used for the hand-washing, wherein the controller may correct the criteria based on the water temperature.

The temperature of the washed hand is affected by the water temperature of water used for hand-washing. Thus, the water temperature of water used for hand-washing is obtained, and the criteria are corrected so as to reduce the influence of the water temperature. For example, when cold water is used, a reference temperature can be corrected to a lower temperature than in the use of warm water.

The drying system may further include a fourth sensor configured to generate timing data indicating the timing of supply of water and soap during the hand-washing, wherein the controller may determine whether to provide the dry medium also based on the timing data.

For example, whether hand-washing with soap is sufficient can be estimated by measuring a time from the timing of soap supply to the timing of water supply. The combined use can further improve the accuracy of determination.

Embodiments of the present disclosure will be described below in accordance with the accompanying drawings. The configurations of the following embodiments are exemplary, and the present disclosure is not limited to the configurations of the embodiments.

### First Embodiment

Referring to FIG. 1, the outline of a drying system according to a first embodiment will be described below. The drying system according to the present embodiment includes a dryer 10. The dryer 10 is an appliance (hand dryer) installed at a washstand in, for example, a powder room in order to dry hands with warm air.

The dryer 10 includes a microcomputer 100 for controlling the dryer, a sensor 200 for sensing an inserted hand, and a drying unit 300 for generating warm air. The microcomputer 100 generates an operation command for the drying unit 300 based on a hand-sensing result and outputs the command. In the present embodiment, the microcomputer 100 determines whether the temperatures of fingers have been sufficiently reduced by a water flow of hand-washing based on sensor data acquired from the sensor 200, and operates the drying unit 300 when the temperatures of the fingers are sufficiently reduced (in other words, hand-washing can be regarded as being correct).

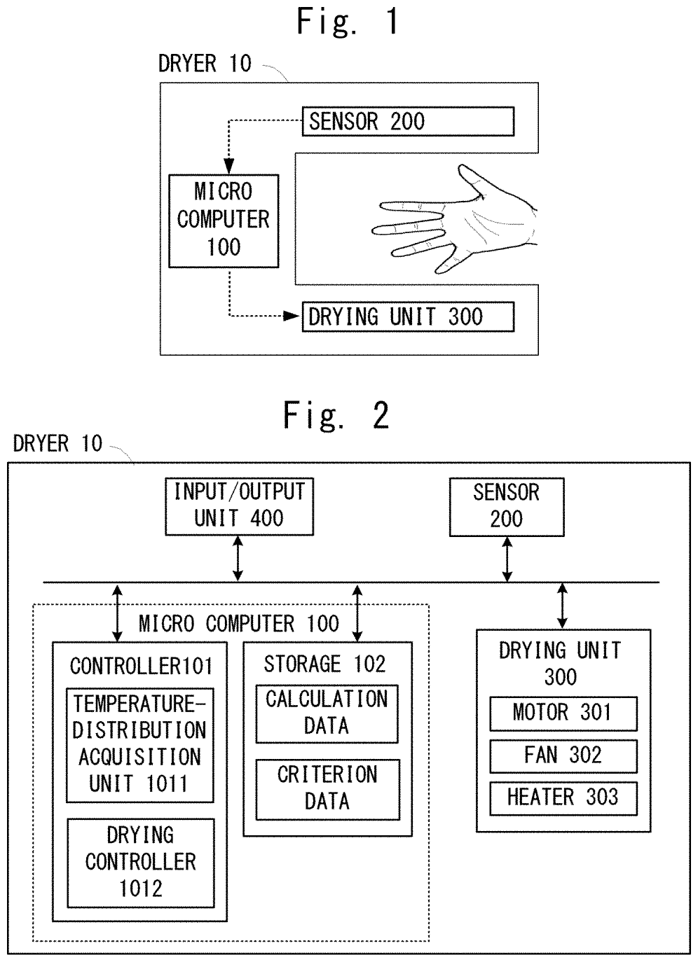

FIG. 2 is a detail diagram illustrating the components of the dryer 10 according to the present embodiment.

The dryer 10 includes the microcomputer 100, the sensor 200, the drying unit 300, and an input/output unit 400.

The microcomputer 100 is a one-chip microcomputer, a package of an arithmetic unit, a main storage, and an auxiliary storage. The arithmetic unit in the present embodiment is a one-chip microcomputer. A unit corresponding to the microcomputer 100 may include a general-purpose computer. Specifically, the microcomputer 100 may be a computer including a processor, e.g., a CPU or a GPU, a main storage, e.g., RAM or ROM, an EPROM, a hard disk drive, and auxiliary storages such as a removable medium. The auxiliary storage stores, for example, an operating system (OS), various programs, and various tables. The stored programs are loaded into the work area of the main storage and are executed therein, and the components or the like are controlled by executing the programs. This can achieve functions for a predetermined purpose as will be described later. However, at least some of or all of the functions may be implemented by a hardware circuit, e.g., an ASIC or an FPGA.

The microcomputer 100 includes a controller 101 and a storage 102.

The controller 101 is an arithmetic unit for the control performed by the microcomputer 100. The controller 101 can be implemented by an arithmetic processing unit, e.g., a CPU.

The controller 101 includes two function modules: a temperature-distribution acquisition unit 1011 and a drying controller 1012. The function modules may be implemented by executing the stored programs by means of a CPU.

The temperature-distribution acquisition unit 1011 acquires the distribution of the surface temperatures of a hand inserted into the dryer 10 (hereinafter, will be simply referred to as a temperature distribution or a hand temperature distribution), based on sensor data acquired from the sensor 200. The sensor data and the temperature distribution will be described later. The acquired temperature distribution is transmitted to the drying controller 1012.

The drying controller 1012 calculates a value (evaluation value) indicating a reduction in finger temperature through hand-washing based on the temperature distribution transmitted from the temperature-distribution acquisition unit 1011. When it is determined that the evaluation value exceeds a threshold value (in other words, the temperatures of fingers have been sufficiently reduced by a water flow), the drying unit 300 is controlled to start a drying operation. A specific method will be described later.

The storage 102 includes a main storage and an auxiliary storage. The main storage is a memory in which programs executed by the controller 101 and data used by the control program is loaded. The auxiliary storage is a device for storing the programs executed by the controller 101 and data used by the control program.

The storage 102 stores data used by the drying controller 1012. In the first embodiment, the storage 102 stores calculation data and reference data.

The calculation data is data for calculating an evaluation value based on the temperature distribution acquired by the temperature-distribution acquisition unit 1011. Furthermore, the reference data includes a threshold value to be compared with the calculated evaluation value. For example, when the calculated evaluation value exceeds the threshold value defined by the reference data, a sufficient reduction in finger temperature can be determined.

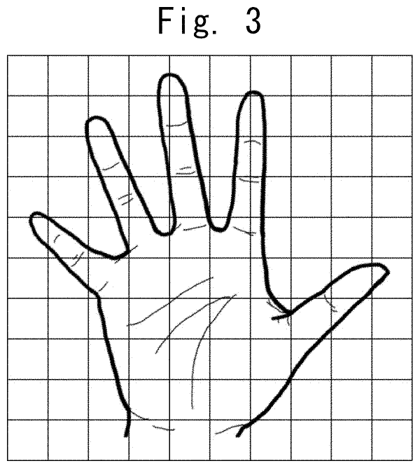

The sensor 200 is a sensor for acquiring the temperature distribution of a hand inserted into the dryer 10. The sensor 200 includes, for example, an element capable of detecting far infrared radiation. The energy of far infrared radiation from an object increases with temperature. Thus, the temperature of an object (a user's finger in the present embodiment) can be measured by detecting the intensity of far infrared radiation by means of the sensor 200. The sensor 200 generates, as sensor data, data indicating the detected intensity of far infrared radiation in the bitmap format and transmits the data to the controller 101. FIG. 3 illustrates a hand inserted into the dryer 10 and a generated bitmap. In this case, a 10-by-10 bitmap is generated. The resolution of the bitmap is not limited to that of FIG. 3.

The drying unit 300 is a unit for drying hands inserted into the dryer 10 (that is, washed wet hands). In the present embodiment, the drying unit 300 includes a motor 301, a rotary fan 302 fixed to the rotating shaft of the motor, and a heater 303 installed in an exhaust passage. The motor 301 rotates the fan 302 to generate an air flow, and the generated air flow is heated by the heater 303, thereby generating a jet of warm air. The operations of the drying unit 300 are controlled by the drying controller 1012.

The input/output unit 400 is an interface device for providing information for a user. The input/output unit 400 includes, for example, a display device and speakers.

A method of controlling the drying unit 300 by the controller 101 based on the acquired hand temperature distribution will be described below.

When the user inserts a hand into the dryer 10, the sensor 200 detects the insertion of the hand and generates sensor data (a bitmap indicating the intensity of far infrared radiation) for calculating the temperature distribution. The generated sensor data is acquired by the temperature-distribution acquisition unit 1011, and then the hand temperature distribution is calculated by a predetermined method.

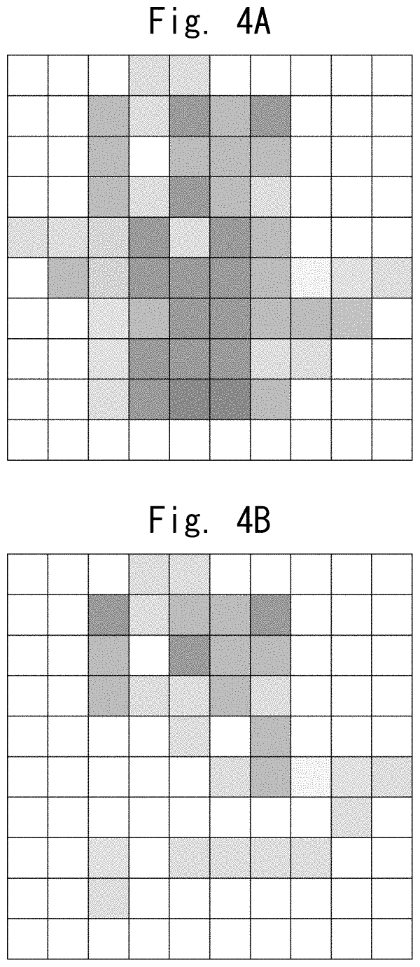

FIG. 4A illustrates an example of the calculated temperature distribution. In the present example, a dark color indicates a low temperature. Hand-washing with running water brings the surface temperature of a hand close to a water temperature with the passage of time. Thus, when a reduction in surface temperature is recognized over a wider range and the surface temperature approaches the water temperature, it can be estimated that hand-washing has been performed for a sufficient time. In the case of an insufficient hand-washing time, as illustrated in FIG. 4B, a reduction in surface temperature is limited. When only some of the fingers are washed, the surface temperature decreases within a limited range.

Thus, whether hand-washing is sufficient or not can be evaluated according to two evaluation criteria: “the range of a decreasing surface temperature” and “a reduction in surface temperature.” The former will be referred to as a first evaluation criterion, and the latter will be referred to as a second evaluation criterion.

In the present embodiment, the drying controller 1012 evaluates the state of the user's hand according to the evaluation criteria and operates the drying unit 300 when the conditions are satisfied. When the conditions are not satisfied, the user is notified of the fact via the input/output unit 400.

As described above, an evaluation can be pertained based on the first evaluation criterion and the second evaluation criterion. For example, a value indicating a difference between a reference temperature (for example, the standard surface temperature of a human hand) and the observed surface temperature of a hand is calculated for each pixel, and the sum of the values of all pixels can be regarded as an evaluation value E. In Expression (1), T_{std} is the standard surface temperature of a human hand, T_{(x,y)} is a surface temperature observed at a corresponding pixel (x,y).

Evaluation value E=Σ(T_{std}−T_{(x,y)}) Expression (1)

According to the illustrated method, the obtained evaluation value increases as the surface temperature of a hand decreases before and after hand-washing, and the obtained evaluation value increases with the extension of an area where a surface temperature decreases. In other words, hand-washing with greater care can be estimated as the obtained evaluation value increases.

Expressions and tables for determining the evaluation value can be stored as calculation data in the storage 102. The threshold value for determining whether the evaluation value satisfies the criterion or not can be also stored as reference data in the storage 102.

In this case, the evaluation value is determined according to Expression (1). Other methods may be used for the evaluation value. For example, the number of pixels including surface temperatures lower than T_{std} may be determined. The number of pixels serves as an evaluation value corresponding to the first evaluation criterion. Alternatively, a representative value of the surface temperatures of an entire hand may be determined, and then a difference from T_{std} may be determined. The difference serves as an evaluation value corresponding to the second evaluation criterion. Furthermore, a predetermined operation may be performed on these evaluation values so as to obtain a final evaluation value E.

The size of the bitmap as sensor data may be normalized in order to suppress the influence of variations in hand size among individuals.

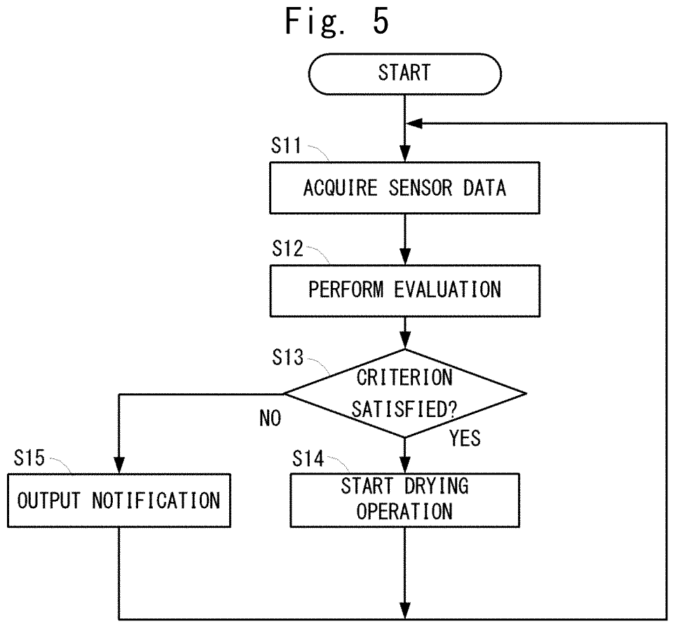

Referring to FIG. 5, processing performed by the controller 101 will be more specifically described below. The processing in the diagram is started when the sensor 200 detects a user's hand.

First, in step S11, the temperature-distribution acquisition unit 1011 acquires the sensor data transmitted from the sensor 200 and calculates the temperature distribution of a hand surface. The calculated temperature distribution is transmitted to the drying controller 1012.

Subsequently, in step S12, the drying controller 1012 performs an evaluation based on the received temperature distribution. In this step, for example, an evaluation value is calculated by using the calculation data.

In step S13, the drying controller 1012 then determines whether the evaluation value calculated in step S12 satisfies the criterion or not according to the reference data.

When the calculated evaluation value satisfies the criterion, the processing advances to step S14, and the drying controller 1012 transmits a control signal to the drying unit 300, starting a drying operation. The drying operation can be continued while the user's hand is detected by the sensor 200.

When the calculated evaluation value does not satisfy the criterion, the processing advances to step S15, and the drying controller 1012 outputs a notification about the unsatisfied criterion via the input/output unit 400. When the first evaluation criterion and the second evaluation criterion are separately evaluated and one of the criteria is not satisfied, a notification about the unsatisfied criterion may be outputted. For example, a notification “only a part of the hand is washed” or “the surface temperature of the hand is not sufficiently reduced” may be provided. Furthermore, a visualized image of the temperature distribution of a hand surface may be generated, and the image may be outputted via the input/output unit 400. Alternatively, an image indicating an ideal temperature distribution may be generated, and the image may be outputted in a comparable foam.

As described above, the dryer 10 according to the first embodiment acquires the temperature distribution of the surface of a washed user's hand and controls a drying operation based on whether the temperature distribution satisfies the criterion. This configuration can encourage a user to wash hands for a sufficient time.

### Second Embodiment

In the first embodiment, the acquired hand-temperature distribution is compared with the standard surface temperature of a human hand. However, the surface temperature of a hand varies among individuals and is affected by environmental factors (including an air temperature and room temperature). In order to address the problem in a second embodiment, the surface temperature of a hand is acquired before hand-washing and is used for comparison.

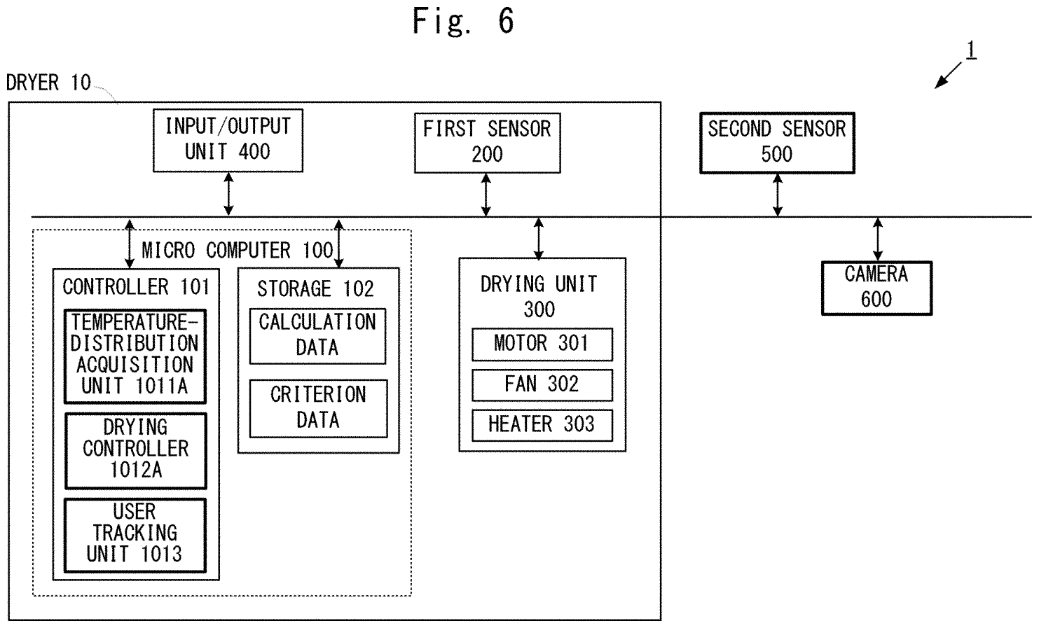

FIG. 6 is a schematic diagram illustrating the configuration of a drying system 1 according to the second embodiment. In the second embodiment, a second sensor 500 and a camera 600 are connected to a dryer 10. In the present embodiment, a sensor 200 will be referred to as a first sensor 200, which is discriminated from the second sensor 500.

The dryer 10 (controller 101) according to the second embodiment further includes a user tracking unit 1013. Differences of the functions of a temperature-distribution acquisition unit 1011A and a drying controller 1012A will be described below.

The second sensor 500 is a sensor for sensing a hand of a user before hand-washing. The second sensor 500 includes the same functions as the first sensor 200. The second sensor 500 is installed near, for example, a washstand for hand-washing. The second sensor 500 may also act as a sensor for an automatic water tap. This configuration can sense a user's hand before turning on a tap.

Sensor data acquired by the second sensor 500 is transmitted to a controller 101 (temperature-distribution acquisition unit 1011A (will be described later)).

The camera 600 is a camera for capturing an image of a user during hand-washing. The camera 600 is preferably located so as to capture an image of the user facing a washstand and an image of the user facing the dryer 10. The images captured by the camera 600 are transmitted to the controller 101 (user tracking unit 1013 (will be described later)).

The temperature-distribution acquisition unit 1011A of the second embodiment is different from the temperature-distribution acquisition unit 1011 of the first embodiment in the additional function of acquiring the temperature distribution of a user's hand surface before hand-washing based on the sensor data acquired from the second sensor 500.

The drying controller 1012A of the second embodiment is different from the drying controller 1012 of the first embodiment in that the temperature distribution of a user's hand surface before hand-washing and the temperature distribution of a washed user's hand surface are compared with each other.

As in the first embodiment, when it is determined that the temperatures of fingers have been sufficiently reduced, the drying controller 1012A controls a drying unit 300 so as to start a drying operation.

The user tracking unit 1013 detects a person included in an image captured by the camera 600 and tracks the position of the person in the image. Furthermore, the user tracking unit 1013 detects a state in which (1) a user stretches the hands toward the second sensor 500 or (2) the same user stretches the hands toward the first sensor 200. Persons can be detected and tracked by known techniques.

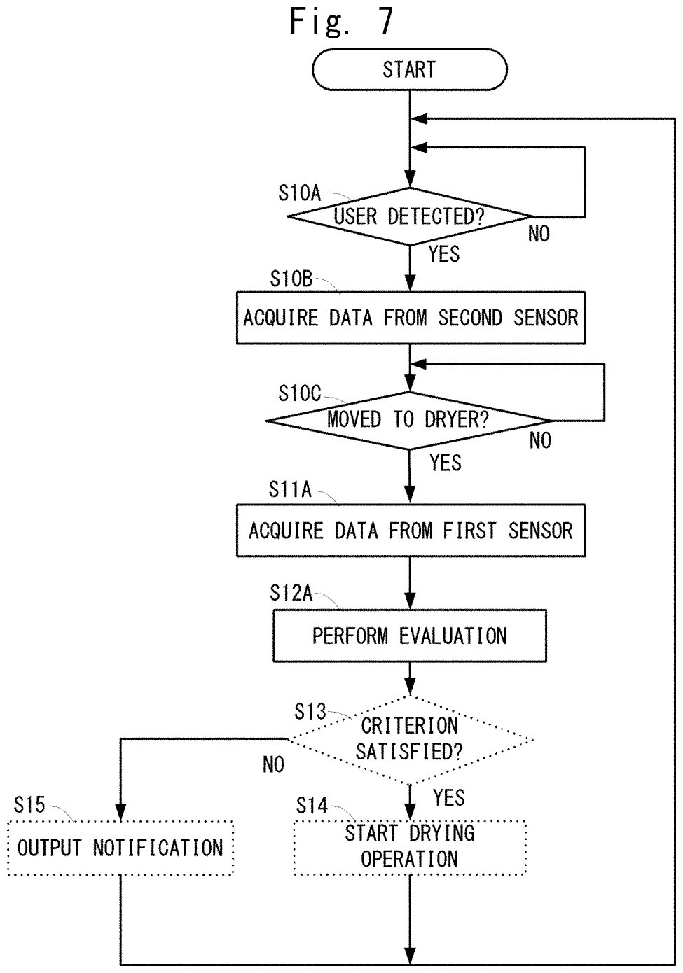

FIG. 7 is a flowchart of processing performed by the controller 101 according to the second embodiment. The indicated processing is periodically performed during a system operation.

First, in step S10A, the user tracking unit 1013 detects a user by analyzing an image transmitted from the camera 600 and determines whether the detected user is stretching the hands toward the second sensor 500 or not. In the case of NO, the processing is repeated until YES is determined. In the case of YES, the processing advances to step S10B.

In step S10B, the temperature-distribution acquisition unit 1011A acquires sensor data (hereinafter, will be referred to as second sensor data) transmitted from the second sensor 500. The acquired second sensor data is temporarily stored.

In step S10C, the user tracking unit 1013 detects the user by analyzing an image transmitted from the camera 600 and determines whether the detected user is stretching the hands toward the first sensor 200 (that is, the dryer 10) or not. In the case of NO, the processing is repeated until YES is determined. In the case of YES, the processing advances to step S11A.

In step S11A, the temperature-distribution acquisition unit 1011A acquires sensor data (hereinafter, will be referred to as first sensor data) transmitted from the first sensor 200. The acquired first sensor data is temporarily stored.

Subsequently, in step S12A, the temperature-distribution acquisition unit 1011A performs an evaluation based on the stored first sensor data and second sensor data.

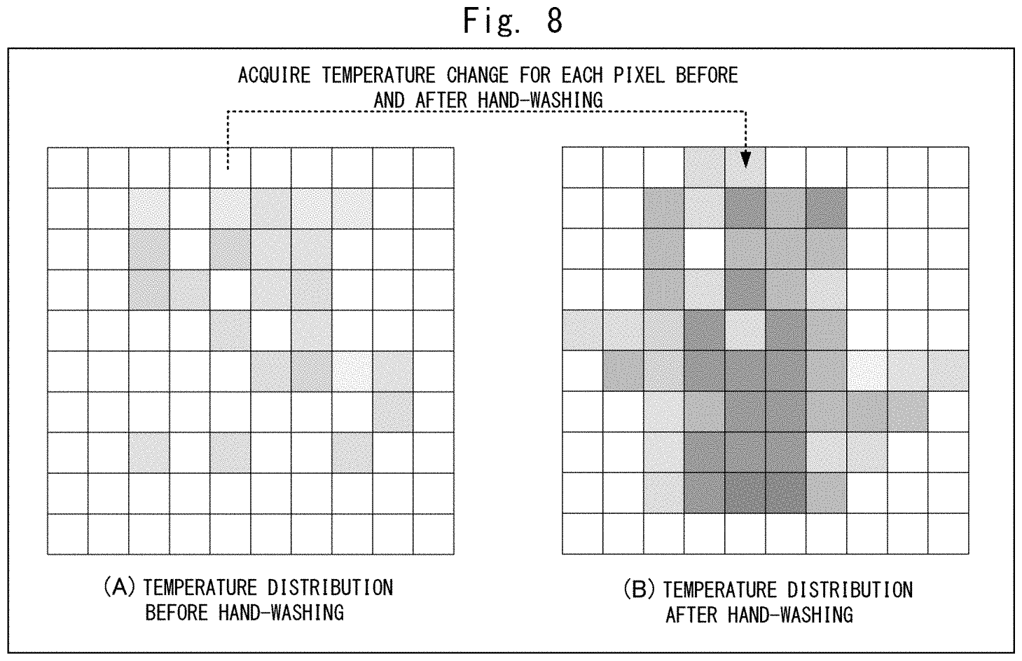

In the first embodiment, the standard surface temperature of a human hand is used as a comparative subject for calculating a reduction in the surface temperature of a hand. In the second embodiment, the temperature distribution obtained based on the second sensor data is used as a comparative subject. Specifically, as indicated in FIG. 8, an actual temperature change is determined for each pixel before and after hand-washing, and then an evaluation value is calculated based on the amount of change.

Expression (2) is an example of a computational expression for an evaluation value E according to the second embodiment. In Expression (2), T_{before (x,y)} indicates a surface temperature of a hand before hand-washing, and T_{after (x,y)} indicates a surface temperature of a hand after hand-washing.

Evaluation value E=Σ(T_{before (x,y)}−T_{after (x,y)}) Expression (2)

Whether the evaluation value E satisfies the criterion or not is determined as in the first embodiment.

As described above, the dryer 10 according to the second embodiment acquires the temperature distribution of a user's hand surface before and after hand-washing and evaluates whether hand-washing is sufficient or not based on a change of the temperature distribution. This configuration can reduce the influence of variations in body temperature among users.

### Third Embodiment

The first and second embodiments can determine whether hand-washing in a water flow is sufficient or not. However, when soap is not used or scrubbing with soap is inadequate, hand-washing is not to be evaluated to be sufficient. A third embodiment improves the accuracy of determination by sensing the supply of water and soap.

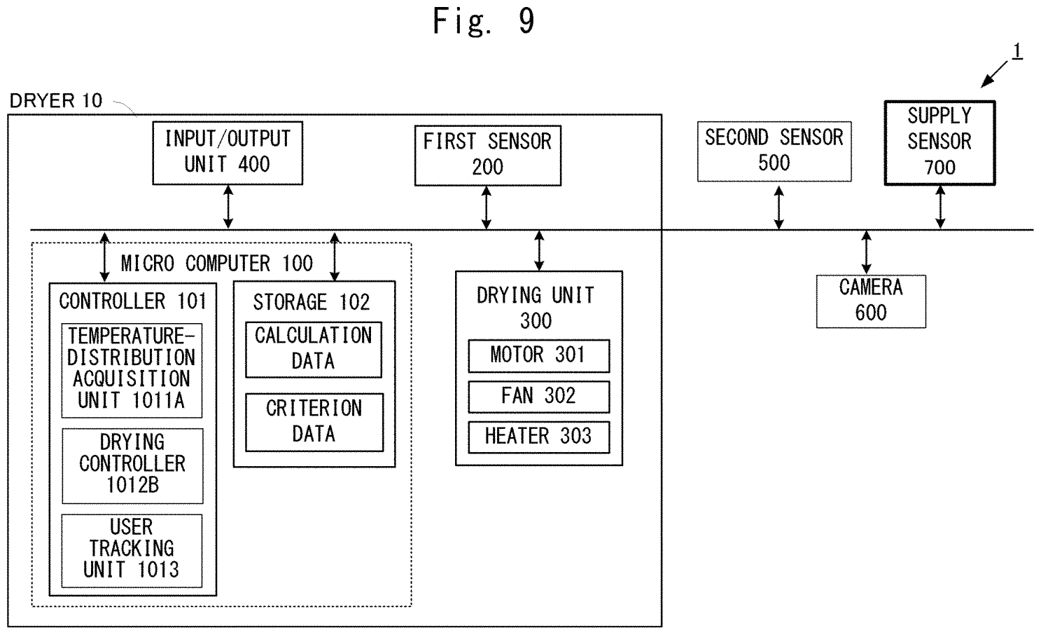

FIG. 9 is a schematic diagram illustrating the configuration of a drying system 1 according to the third embodiment. The drying system 1 according to the third embodiment is configured such that a supply sensor 700 for sensing the supply of water and soap is added to the drying system 1 according to the second embodiment. The second sensor 500 and the supply sensor 700 are installed at the same washstand.

The supply sensor 700 is a sensor for outputting time-series data (hereinafter, will be referred to as timing data) on the timing of supply of water and soap (or liquid soap). The supply sensor 700 is installed along with, for example, an automatic water tap for supplying water and soap at a washstand.

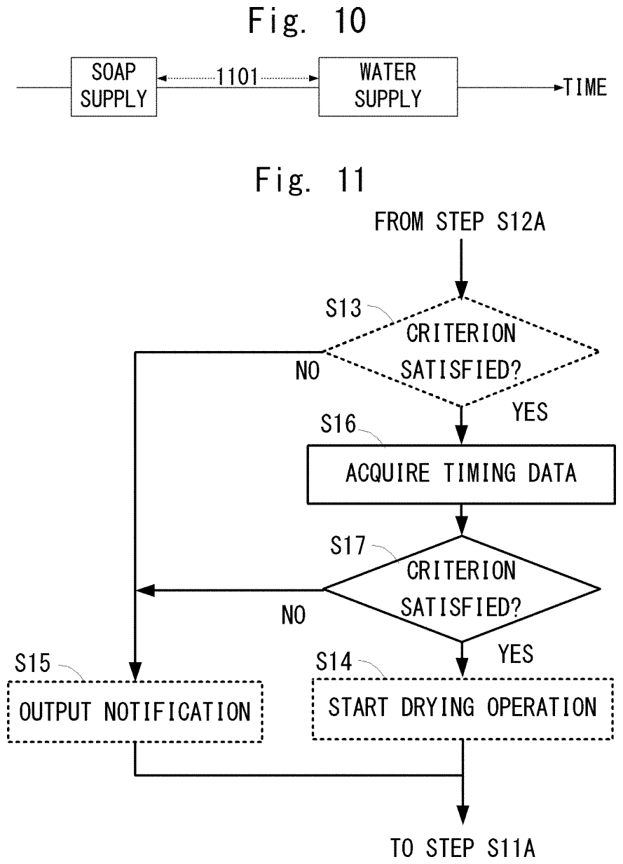

FIG. 10 is an explanatory drawing of the timing data outputted by the supply sensor 700. As illustrated in FIG. 10, the timing data includes the timing of soap supply and the timing of water supply. For example, when an interval indicated by reference numeral 1101 is shorter than a predetermined threshold value or the interval is not present, hand-washing with soap is estimated to be insufficient.

A drying controller 1012B is different from the drying controller 1012A of the second embodiment in the additional function of determining whether hand-washing with soap is sufficient or not based on the timing data.

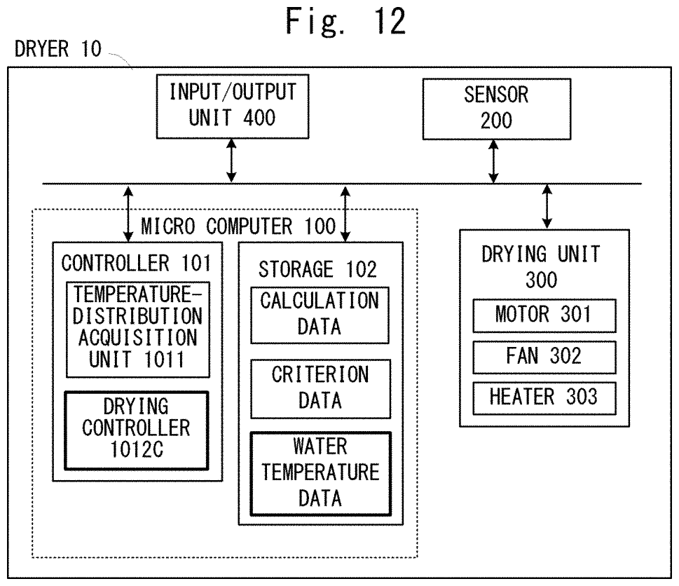

FIG. 11 is a part of a flowchart of processing performed by a controller 101 according to the third embodiment.

In the third embodiment, the drying controller 1012B having performed the processing of step S13 acquires timing data in a past predetermined period from the supply sensor 700 corresponding to a washstand used by a user (step S16). Moreover, whether a sufficient interval is placed between the timing of soap supply and the timing of water supply (that is, whether a sufficient time is maintained for hand-washing with soap) is determined based on the timing data (step S17). When the criterion is satisfied, the process advances to step S14 to start a drying operation. When the criterion is not satisfied, the process advances to step S15 to output a notification about the unsatisfied criterion via the input/output unit 400. When the criterion is not satisfied in one of steps S13 and S17, a notification about the unsatisfied criterion may be provided. For example, a notification “the surface temperature of the hand is not sufficiently reduced” or “an insufficient time for hand-washing with soap” may be provided. Other steps are similar to those of the second embodiment.

As described above, the third embodiment determines whether hand-washing is sufficient or not by using data on the records of soap supply. This configuration can encourage a user to wash the hands with soap.

### Fourth Embodiment

A reduction in the surface temperature of a hand through hand-washing depends upon the water temperature. For example, using warm water leads to a smaller reduction in the surface temperature of a hand than in the use of cold water. Thus, whether hand-washing is sufficient or not may be incorrectly evaluated at some water temperatures. In order to address the problem in a fourth embodiment, whether hand-washing is sufficient or not is determined in consideration of a water temperature.

FIG. 12 is a schematic diagram illustrating the configuration of a dryer 10 according to the fourth embodiment. The dryer 10 according to the fourth embodiment is configured such that water temperature data for defining a water temperature is further stored in the dryer 10 according to the first embodiment.

A drying controller 1012C of the fourth embodiment is different from the drying controller 1012 of the first embodiment in the additional function of correcting reference data based on a water temperature.

The reference data can be corrected in, for example, step S12. For example, in the use of warm water for hand-washing, a threshold value used in step S13 is corrected to a smaller threshold value than in the use of cold water. In the use of cold water for hand-washing, the threshold value is corrected to a larger threshold value than in the use of warm water. Data for correction (for example, expressions and tables that indicate the relationship between a water temperature and a correction of the threshold value) may be stored in advance in a storage 102.

As described above, the dryer 10 according to the fourth embodiment defines the water temperature of water for hand-washing in advance and corrects the threshold value serving as a criterion. This configuration can reduce the influence of a water temperature.

In the present example, the water temperature data is stored in advance. When a sensor (third sensor) for sensing a water temperature is available, a water temperature may be acquired each time based on sensor data acquired from the sensor.

### Modification

The foregoing embodiments are merely exemplary, and the present disclosure can be changed as necessary without departing from the scope of the disclosure.

For example, the processing and units described in the present disclosure may be implemented in any combination unless a technical contradiction arises.

In the descriptions of the embodiments, an evaluation value is calculated. A similarity between a reference temperature distribution and an acquired temperature distribution may be calculated instead of an evaluation value, and whether a reduction in finger temperature is sufficient or not may be determined based on the similarity.

In the description of the embodiments, the dry medium is warm air. The dry medium may be any medium other than air. For example, the drying unit 300 may be replaced with a mechanism for supplying paper towels or a mechanism for supplying cloth towels.

Processing to be pertained by a single apparatus may be shared among multiple apparatuses. Alternatively, processing to be performed by different apparatuses may be performed by a single apparatus. In a computer system, the hardware configuration (server configuration) of functions can be flexibly changed.

The present disclosure can be also implemented by providing a computer with a computer program including the functions of the foregoing embodiments and executing the program read by at least one processor included in the computer. Such a computer program may be provided for the computer by a non-temporary computer-readable storage medium that is connectable to the system bus of the computer or may be provided for the computer via a network. Such non-temporary computer-readable storage media include, for example, any types of disks such as a magnetic disk (a floppy (registered trademark) disk or a hard disk drive (HDD)) and an optical disc (a CD-ROM or a DVD disc/Blu-ray Disc), read-only memory (ROM), random access memory (RAM), EPROM, EEPROM, a magnetic card, a flash memory, an optical card, and any types of media suitable for storing electronic commands.

What is claimed is:

1. A drying system comprising:
a first sensor configured to sense a washed hand of a user; and
a controller including at least one processor configured to acquire first information on a temperature of the washed hand of the user based on data acquired from the first sensor and
determine whether to provide a dry medium for the user based on the first information.

2. The drying system according to claim 1, wherein the controller determines that the dry medium is to be provided when the temperature of the hand is lower than a predetermined value.

3. The drying system according to claim 1, wherein the first sensor is a sensor capable of acquiring a temperature distribution of the hand,
the first information is information on the temperature distribution of the hand, and
the controller determines whether to provide the dry medium based on the temperature distribution of the hand.

4. The drying system according to claim 3, wherein the controller determines that the dry medium is to be provided when an evaluation value calculated from the temperature distribution of the hand satisfies a predetermined threshold value.

5. The drying system according to claim 1, further comprising a second sensor configured to sense the hand of the user before the hand-washing,
wherein the controller acquires second information on a temperature of the hand of the user before the hand-washing, based on data acquired from the second sensor, and
determines whether to provide the dry medium based on both of the first information and the second information.

6. The drying system according to claim 5, wherein the first and second sensors are sensors capable of acquiring a temperature distribution of the hand,
the first and second information is information on the temperature distribution of the hand, and
the controller determines whether to provide the dry medium based on a change of the temperature distribution of the hand.

7. The drying system according to claim 1, further comprising a storage configured to store criteria for determining whether to provide the dry medium.

8. The drying system according to claim 7, further comprising a third sensor configured to obtain a water temperature of water used for the hand-washing,
wherein the controller corrects the criteria based on the water temperature.

9. The drying system according to claim 1, further comprising a fourth sensor configured to generate timing data indicating timing of supply of water and soap during the hand-washing,
wherein the controller determines whether to provide the dry medium also based on the timing data.

10. An information processing apparatus comprising a controller including at least one processor configured to acquire first information on a temperature of a washed hand of a user based on data acquired from a first sensor configured to sense the washed hand of the user, and
determine whether to provide a dry medium for the user based on the first information.

11. The information processing apparatus according to claim 10, wherein the controller determines that the dry medium is to be provided when the temperature of the hand is lower than a predetermined value.

12. The information processing apparatus according to claim 10, wherein the first sensor is a sensor capable of acquiring a temperature distribution of the hand,
the first information is information on the temperature distribution of the hand, and
the controller determines whether to provide the dry medium based on the temperature distribution of the hand.

13. The information processing apparatus according to claim 12, wherein the controller determines that the dry medium is to be provided when an evaluation value calculated from the temperature distribution of the hand satisfies a predetermined threshold value.

14. The information processing apparatus according to claim 10, wherein the controller acquires second information on a temperature of the hand of the user before the hand-washing, based on data acquired from a second sensor configured to sense the hand of the user before the hand-washing, and
determines whether to provide the dry medium based on both of the first information and the second information.

15. The information processing apparatus according to claim 14, wherein the first and second sensors are sensors capable of acquiring a temperature distribution of the hand,
the first and second information is information on the temperature distribution of the hand, and
the controller determines whether to provide the dry medium based on a change of the temperature distribution of the hand.

16. The information processing apparatus according to claim 10, further comprising a storage configured to store criteria for determining whether to provide the dry medium.

17. The information processing apparatus according to claim 16, wherein the controller further acquires a water temperature of water used for the hand-washing and corrects the criteria based on the water temperature.

18. The information processing apparatus according to claim 10, wherein the controller further acquires timing data indicating timing of supply of water and soap during the hand-washing, and
the controller determines whether to provide the dry medium also based on the timing data.

19. A non-temporary storage medium for storing a program for causing an information processing apparatus to acquire first information on a temperature of a washed hand of a user based on data acquired from a first sensor configured to sense the washed hand of the user, and
determine whether to provide a dry medium for the user based on the first information.

20. The non-temporary storage medium for storing a program according to claim 19, wherein the first sensor is a sensor capable of acquiring a temperature distribution of the hand,
the first information is information on the temperature distribution of the hand, and
whether to provide the dry medium is determined based on the temperature distribution of the hand.
